(19) United States
(12) Patent Application Publication
(10) Pub. No.: US 2025/0235654 A1
(43) Pub. Date: Jul. 24, 2025

(54) FRAME ASSEMBLY IN A FIELD OF FOREHEAD SUPPORT

(71) Applicant: DCSTAR INC, New York, NY (US)

(72) Inventor: David LUO, New York, NY (US)

(73) Assignee: DCSTAR INC, New York, NY (US)

(21) Appl. No.: 18/416,125

(22) Filed: Jan. 18, 2024

(51) Int. Cl. A61M 16/06

(52) U.S. Cl. CPC A61M 16/0683; A61M 2205/0288

(57) ABSTRACT

A frame assembly with forehead support includes an elbow, a forehead support frame, a cushion pad, an exhaust port, and fixing parts. The forehead support frame includes a first joint, a second joint, a support rod and a forehead pad to connect to the support rod. The first joint forms a detachable connection with the elbow. The forehead support frame further includes connecting end parts on both sides to connect to fixing parts. Magnetic buckles and connecting end parts are configured to connect to and secure a headgear. The cushion pad is configured to be placed on the forehead pad, providing cushioning to a face during use. Additionally, an exhaust port can be set on the elbow.

### TECHNICAL FIELD

The disclosure pertains to a frame assembly in the field of forehead support, which is applicable for one or more of treatments and improvements of respiratory system diseases. It mainly includes an elbow, a forehead support frame, a cushion pad, an exhaust port, and fixing parts.

### BACKGROUND

Sleep apnea syndrome (SAS), also known as sleep-disordered breathing, is a common sleep-related breathing disorder disease, generally categorized into central sleep apnea syndrome (CSAS), obstructive sleep apnea syndrome (OSAS), and mixed sleep apnea syndrome (MSAS). Obstructive sleep apnea primarily affects the pharyngeal area. During sleep, the size of the pharyngeal cavity is primarily regulated by the contraction of surrounding muscles. Factors like thickened pharyngeal walls, enlarged tonsils and tongue, an enlarged soft palate, retracted mandible, and edema of the epiglottis can lead to narrowing or blockage of the pharyngeal cavity, thus causing obstructive sleep apnea. Sleep apnea syndrome is typically diagnosed if there are more than 30 episodes of apnea and hypopnea per 7 hours of sleep, or if the apnea-hypopnea index is greater or equal to 5 times per hour. Obstructive sleep apnea accounts for about 90% of the snoring population. It is mainly characterized by snoring with irregular snore sounds during nighttime sleep, repeated apnea and awakening, significant daytime sleepiness, and reduced memory. Obstructive sleep apnea is characterized by apnea and hypopnea due to pharyngeal narrowing or blockage during sleep. It has a high prevalence and is a potentially lethal disorder with complications. It can lead to various diseases, such as coronary heart disease, hypertension, diabetes, stroke, myocardial ischemia, myocardial infarction, and others. The treatment of obstructive sleep apnea primarily involves increasing the tension of the pharyngeal airway, expanding the airway volume, and establishing a bypass for ventilation to eliminate apnea and hypopnea. The general treatment has five methods, including improving lifestyle habits, medication, airway positive pressure ventilation treatment (including continuous positive airway pressure (CPAP), automatic continuous positive airway pressure, and bilevel positive airway pressure), oral correction, and surgical treatment. Among these, CPAP often treats moderate to severe sleep apnea patients and is also a recommended way to treat sleep apnea, as it can immediately improve sleep quality and reduce the discomfort and complications associated with surgery. Statistics show that 60%-75% of patients are satisfied with CPAP treatment, but only 46% of patients use it regularly after long-term follow-up. The beneficial effects of CPAP depend on long-term use, and the standard frequency of using the ventilator is more than 5 days a week and at least 4 hours each night to reach treatment effect. Considering the long duration and long treatment of CPAP therapy, the comfort and convenience of the treatment components for obstructive sleep apnea are critical for patients. Treatment components for obstructive sleep apnea typically include the patient interface pad, frame, air tube, ventilator, elbow, and headgear.

The market has introduced frames with forehead supports, which offer enhanced stability compared to frames without forehead support. These forehead support frames distribute pressure caused by treatment components on the face, reducing the sense of pressure concentrated in facial areas and providing patients with a more stable, comfortable, and effective treatment process. Although there are various styles and types of forehead support frames on the existing market, none fully meet the diverse needs of patients. Some forehead support frames, made with rigid materials, can cause discomfort when in contact with the patient's face. Other forehead support frames have weak adaptability, and their fixed configurations are not the best option for patients. Additionally, some forehead support frames have extremely complex structures, which are not conducive to production. They increase labor and material costs, potentially leading to waste and environmental impacts. For complex forehead support frames, despite their multiple functions, supply chain issues can arise in emergencies, leading to prolonged stock shortages.

### SUMMARY

The objective of this disclosure is to provide a novel forehead support frame component that is modular, has excellent comprehensive performance, is low-cost, and has a simple structure. It aims to overcome the limitations of existing similar products in current technology. Thereby providing a more effective method than the existing technology, with broader application scenarios and space, and an easier and more feasible processing method to fix the patient's interface pad and assist in sealing and delivering the pressurized gas output from the ventilator to the patient's airway to provide treatment for sleep apnea.

The disclosure relates to a frame assembly in a field of forehead support, mainly involving the frame, elbow, and patient interface pad. In the treatment of obstructive sleep apnea, the frame connects the elbow and patient interface pad, and then by connecting the headgear, the frame provides support and stability to other components, ensuring the mask fits correctly on the patient's face. This helps prevent movement or loosening of the patient interface pad during the night, which can cause air leakage, therefore increasing treatment effectiveness.

This disclosure simplifies the structure of the forehead support frame without sacrificing its functionality. It adds quick-release magnetic buckles that are convenient for users and comfortable cushioning pads. The elbow component is also redesigned, employing a modular design approach in various aspects. This enhances the range of options available to users, as well as improving cost-effectiveness and comfort. Compared to existing forehead support frame components, this disclosure offers more advantages and market potential, making it a more competitive product in the field of forehead support frames.

In an embodiment, a frame assembly in a field of forehead support is provided. The frame assembly in the field of forehead support is for use in conjunction with other parts to supply continuous pressurized breathing gas to airways for the treatment of sleep breathing disorders. The frame assembly in a field of forehead support includes: a forehead support frame, including a first joint that forms a detachable connection with an elbow, a second joint away from the elbow, a wall formed between the first joint and the second joint, and a support rod extending upward from the wall between the first joint and the second joint to a forehead, along with a forehead pad connected to the support rod. And the forehead pad is configured to distribute the pressure of the patient interface pad on the face across the forehead during use. The forehead support frame further includes connecting end parts on both sides that extend at an angle towards a side near a face to connect to fixing parts. The entire forehead support frame is made of same material. The elbow includes a first interface with a clamping structure. The elbow forms a detachable connection with the first joint of the forehead support frame through the clamping structure. The elbow is also configured with an exhaust port, configured to allow a continuous flow of exhaled gas to an external environment.

In an embodiment, the frame assembly in a field of forehead support further comprises a cushion pad that forms a detachable connection with the forehead support frame.

In an embodiment, an angle between a straight line where the connecting end parts on both sides of the forehead support frame are located and a parallel line to an axis of the first joint of the forehead support frame is at or between 5 to 90°.

In an embodiment, an angle between the straight line where the connecting end parts on both sides of the forehead support frame are located and a plumb line at its position is at or between 0 to 85°.

In an embodiment, the wall formed between the first joint and the second joint of the forehead support frame comprises a snap-fitting to connect to the patient interface pad.

In another embodiment, a frame assembly in a field of forehead support is provided. The frame assembly in the field of forehead support is for use in conjunction with other parts to supply continuous pressurized breathing gas to patients for the treatment of sleep breathing disorders. The frame assembly in a field of forehead support includes: a forehead support frame, including a first joint that forms a detachable connection with an elbow, a second joint away from the elbow, a wall formed between the first joint and the second joint, and a support rod extending upward from the wall between the first joint and the second joint to a forehead, along with a forehead pad connected to the support rod. And the forehead pad is configured to distribute the pressure of the patient interface pad on the face across the forehead during use. The forehead support frame further includes connecting end parts on both sides that extend at an angle towards a side near a face to connect to fixing parts. A cushion pad comprises a first end part connected to the forehead pad, and a second end part that contacts the face, which is configured to provide a certain cushioning to the face during use. The fixing parts are quick-release magnetic buckles, including the connecting ends that connect to the forehead support frame, and a magnetic attraction end. These quick-release magnetic buckles are configured to have a protruding magnetic attraction structure, allowing the forehead support frame to be releasably connected and detached from a headgear.

In one embodiment, the frame assembly in a field of forehead support further comprises the cushion pad that forms a detachable connection with the forehead support frame.

In an embodiment, an area of the cushion pad in contact with the face is no less than 6 cm².

In an embodiment, a thickness of the cushion pad is at least 3 mm.

In an embodiment, the material of the cushion pad includes one or more of foam material or silicone material.

In another embodiment, a frame assembly in a field of forehead support is provided. The frame assembly in the field of forehead support is for use in conjunction with other parts to supply continuous pressurized breathing gas to patients for the treatment of sleep breathing disorders. The frame assembly in a field of forehead support includes: a forehead support frame, including a first joint that forms a detachable connection with an elbow, a second joint away from the elbow, a wall formed between the first joint and the second joint, and a support rod extending upward from the wall between the first joint and the second joint to a forehead, along with a forehead pad connected to the support rod. And the forehead pad is configured to distribute the pressure of the patient interface pad on the face across the forehead during use. The forehead pad has at least one through opening to accommodate a headgear to form a connection. The forehead support frame further includes connecting end parts on both sides that extend at an angle towards a side near a face to connect to fixing parts. The elbow is configured to receive ventilator airflow that flows through the gas transmission tube, and to deliver the ventilator airflow into the patient interface pad. The elbow has a first interface that forms a detachable connection with the first joint of the forehead support frame and a second interface connected to the gas transmission tube.

In an embodiment, the frame assembly in a field of forehead support further comprises a cushion pad that forms a detachable connection with the forehead support frame.

In an embodiment, the second interface of the elbow is configured to connect to the gas transmission tube.

In an embodiment, a height of the support rod is at or between 3 to 12 cm. In an embodiment, an angle between a line connecting both ends of the support rod on the frame assembly in a field of forehead support and a plumb line at its position is at or between 3 to 45°.

In another embodiment, a frame assembly in a field of forehead support is provided. The frame assembly in the field of forehead support is for use in conjunction with other parts to supply continuous pressurized breathing gas to patients for the treatment of sleep breathing disorders. The frame assembly in a field of forehead support includes: a forehead support frame, including a first joint that forms a detachable connection with an elbow, a second joint away from the elbow, a wall formed between the first joint and the second joint, and a support rod extending upward from the wall between the first joint and the second joint to a forehead, along with a forehead pad connected to the support rod. And the forehead pad is configured to distribute the pressure of the patient interface pad on the face across the forehead during use. The forehead pad has at least one through opening to accommodate a headgear to form a connection. The forehead support frame further includes connecting end parts on both sides that extend at an angle towards a side near a face to connect to fixing parts. A cushion pad has a first end part connected to the forehead pad and a second end part that contacts the face. There is a certain distance between the first end part and the second end part, configured to provide facial buffering when the cushion pad is in use. An area of the cushion pad that contacts the face is no less than 6 cm².

In an embodiment, a non-detachable connection is formed between the cushion pad and the forehead pad of the forehead support frame.

In an embodiment, a distance between the first end part and the second end part of the cushion pad is at or between 1 to 5 cm.

In an embodiment, an angle between a straight line where the connecting end parts on both sides of the forehead support frame are located and a parallel line to the axis of the first joint of the forehead support frame at its position is at or between 5 to 90°.

In an embodiment, the forehead pad has an opening that stabilizes and accommodates a part of the cushion pad.

In another embodiment, a frame assembly in a field of forehead support is provided. The frame assembly in the field of forehead support is for use in conjunction with other parts to supply continuous pressurized breathing gas to patients for the treatment of sleep breathing disorders. The frame assembly in a field of forehead support includes: a forehead support frame, including a first joint that forms a detachable connection with an elbow, a second joint away from the elbow, a wall formed between the first joint and the second joint, and a support rod extending upward from the wall between the first joint and the second joint to a forehead, along with a forehead pad connected to the support rod. And the forehead pad is configured to distribute the pressure of the patient interface pad on the face across the forehead during use. The forehead support frame has at least three connection points to connect to a headgear. Two of the connection points are set on connecting end parts that extend at an angle towards the side near the face on both sides of the forehead support frame. The connection points are configured to connect to fixing parts or the headgear. The elbow comprises a first interface with a clamping structure. The elbow forms a detachable connection with the first joint of the forehead support frame through the clamping structure. The elbow is also configured with an exhaust port, configured to allow a continuous flow of exhaled gas to an external environment.

In an embodiment, the frame assembly in a field of forehead support further comprises a cushion pad that forms a detachable connection with the forehead support frame.

In an embodiment, a length of the connecting end parts on both sides of the forehead support frame is at or between 1 to 5 cm.

In an embodiment, an area of the cushion pad that contacts the face is no less than 6 cm².

In an embodiment, an angle between a line connecting both ends of the support rod on the frame assembly in a field of forehead support and a plumb line at its position is at or between 3 to 45°.

Implementing the frame assembly in the field of forehead support in the disclosure provides at least the following beneficial effects:

1. In terms of comprehensive aspects, compared to existing forehead support frames on the market, the forehead support frame disclosed has superior highlights in all aspects. It is more advantageous and belongs to a more competitive frame assembly in a field of forehead support. Referencing the design philosophy from the Tesla automotive design field, the disclosure simplifies the frame and heavily incorporates modular design. The forehead support frame is treated as a separate component. Under the premise of ensuring and optimizing its functionality, the forehead support frame is made more convenient and comfortable. Specifically, a. compared to the existing forehead support frames like the Mirage Activa™ LT Nasal CPAP Mask, ComfortGel Blue, and Ultra Mirage™ II Nasal CPAP Mask, they require connection to larger patient interface pads, appear bulky visually and in use, exert a more serious sense of pressure on the patient, and are likely to cause discomfort. Further, the larger patient interface pad encompasses a greater area of the patient's face, meaning that the discrepancy in level in how the relatively bigger patient interface pad fits around different parts of the face is greater, making it more prone to air leakage and thus reducing the effectiveness of the treatment. In the disclosure, the forehead support frame connects to a smaller patient interface pad, making it more patient-friendly and less prone to air leakage issues. It uses less material and is smaller in size, which makes it easier to carry and transport. It also includes quick-release magnetic buckles at the connection ends where the frame joins the headgear on both sides, making the installation and removal of the frame much simpler and providing a more convenient user experience for patients. The quick-release magnetic buckles are connected to the connecting end parts of the sides of the forehead support frame through a snap-fitting. The buckles have protrusions to store magnets and during connection, the buckles will be connected by the attraction from different magnetic poles on the opposite side. Due to the protruding and connection to corresponding grooves on the other side, they have a baffle in the direction of the force, making them unlikely to fall off. To detach, one simply needs to pull one side slightly in a perpendicular direction for easy removal. This method is time-saving and labor-saving, as the quick-release magnetic buckles are designed for a detachable connection with the connecting end parts on both sides of the forehead support frame. Patients can choose to use different quick-release connectors or not use them at all, according to their preferences or needs, offering a variety of different options. If there is a gas leak in the frame assembly in a field of forehead support, the quick-release design of the magnetic buckle facilitates rapid adjustment of the frame assembly in a field of forehead support by the patient. The design of the quick-release magnetic buckle also makes the disassembly and installation steps more straightforward and clear, which is beneficial for elderly patients or those with less dexterity in their hands to install and remove. The disclosure, compared to the existing forehead support frames mentioned above, reduces the time required for disassembly or installation by at least half. Regarding the overall design of the forehead support frame, the forehead support frame in the disclosure creates a form that is more straightforward, mostly composed of smooth and neat walls. By presenting the forehead support frame as a simpler module, it not only provides advantages in terms of cleaning and patient use but also in production. b. Referencing the existing ResMed Mirage™ FX, Eson™ Nasal CPAP Mask, and Eson™ 2 Nasal CPAP Mask, a part of the forehead support frame where it contacts the patient's forehead in these designs is a rigid part. Although the rigid part is shaped to curve towards the face side to fit the patient's forehead as closely as possible, it is not entirely effective. Since the human forehead is not a perfectly smooth curve, the forehead pad cannot fully conform to the patient's forehead to distribute force for more comfortable use for patients. Furthermore, people have varying degrees of forehead protrusion, and the existing forehead pads of these forehead support frames cannot accommodate the degree of forehead protrusion of different users. When pressure is concentrated on the forehead, some patients may feel very uncomfortable. Additionally, the rigid parts usually do not have the ability to absorb shocks or compression. When the rigid part comes into direct contact with the skin, any external force is directly transmitted to the skin, and the lack of breathability in the rigid parts can easily lead to moisture accumulation, causing discomfort to the patient's skin. The disclosure utilizes a cushion pad placed at the forehead pad area, which on the one hand, adapts to the curve or irregular shape of the forehead. The flexible material evenly distributes the pressure brought about by the headgear fixation, preventing pressure concentration in specific areas, like higher protrusions on the forehead, thereby avoiding pain or discomfort. The flexible material absorbs a certain amount of force, thus reducing the sensation of pressure on the patient's forehead. The gentle touch of the flexible material provides a more comfortable wearing experience for the patient. Furthermore, the present disclosure has quick-release magnetic buckles, which make it more convenient and easy to disassemble and install, saving time and effort. c. The existing Mirage™ FX forehead support frame has a complex structure. Though it offers convenience in terms of functionality, the complex structure and manufacturing process of the Mirage™ FX result in high production and processing costs. It uses silicone and rigid materials and is constructed through a secondary injection molding process. Additionally, its exhaust port is located on the matching patient interface pad. This production method leads to a higher defect rate and increased costs. In special circumstances, the supply of Mirage™ FX may become relatively difficult, leading to prolonged periods of product unavailability. The forehead support frame of this disclosure is entirely made of one material, which simplifies the structure on the premise of ensuring functionality. It mostly consists of smooth walls. Furthermore, the exhaust port of the frame assembly in a field of forehead support of the disclosure is located on the elbow. The elbow forms a detachable connection with the forehead support frame, reducing the defect rate and production cost of the frame assembly in a field of forehead support. This enhances the quality of the product and has a simple processing, preventing stock shortages.

2. The frame assembly in a field of forehead support of this disclosure simplifies the product structure on the premise of ensuring its functional integrity, thereby improving supply chain efficiency, and reducing production costs and risks. This makes the frame assembly in a field of forehead support of the disclosure more competitive in the market. The forehead support frame shown in the disclosure can be broadly divided into four parts. It is integrally injection-molded from one material, and the four parts include, the central part that includes the first joint, the second joint, and the wall connecting the first joint and the second joint, and the central part is configured to connect to the patient interface pad; the forehead pad part; the support rod part connecting the forehead pad with the central part; and the connecting end parts on both sides. The outer walls of all four parts are smooth and uniform without any complex structure such as protrusions or indentations, making the form simple and the volume relatively smaller, allowing for more simple and more efficient production for molds, reducing manpower, equipment, and energy costs during manufacturing. The reduction in costs and increased efficiency allow for quicker adjustments to rapidly respond to and meet changes in market demand, enabling more rapid innovation. The simplicity of the structure makes the production process easier to manage and control, ensuring product quality. Moreover, the reduction in parts and the use of modular components can effectively lower inventory variety, reduce the risk of excess stock, and simplify the complexity of material management. In summary, this disclosure better meets customer needs, enhances product maintainability and life-cycle value, and improves supply chain efficiency. Additionally, the simplified structure leads to reduced material usage and the abandonment rate of waste, positively impacting environmental protection and climate change.

3. The disclosure includes a quick-release elbow that forms a detachable connection with the forehead support pad, modularizing the elbow. Patients can choose different elbows to connect to the forehead support frame according to their preferences and needs. Therefore, different types of patients are allowed to replace different elbows to adapt to varying degrees of airflow pressure and to accommodate tubes of different diameters and lengths. According to some opinions from CPAP forums and Amazon customer reviews, the disclosure understands that different patients have different requirements for the pressure of the airflow entering their airways. Moreover, some patients find a 22 mm gas transmission tube to be somewhat cumbersome and oppressive for the nasal mask. Patients also have varying needs regarding the length of the tube. Therefore, in this disclosure's frame assembly in a field of forehead support, the exhaust port is set on the detachable elbow. If a patient cannot obtain the optimal airflow pressure size from the ventilator to meet their needs, they can adjust the airflow pressure entering their airway by replacing the elbow with different apertures or numbers of exhaust ports. Patients can choose different elbows with different apertures or numbers of exhaust ports according to their own respiratory resistance. For special patients with respiratory dysfunction and respiratory failure, elbows without holes can also be chosen and replaced. In addition to choosing elbows with different apertures or numbers of exhaust ports, patients can also choose elbows with or without noise reduction. Generally, elbows with noise reduction and those without are two different types of elbows. The modular design offers a variety of choices for elbows, allowing users who prefer noise reduction or no noise reduction to purchase and replace them according to their preferences or needs. Additionally, the second interface of the elbow, which connects to the tube, also comes in different sizes to connect to tubes of varying diameters or lengths, such as short tubes or gas transmission tubes. This is intended to allow patients to independently choose the elbow that best suits them, making continuous positive airway pressure (CPAP) therapy more comfortable.

4. The frame assembly in a field of forehead support provided by this disclosure reduces user costs in multiple aspects. Because the forehead support frame of this disclosure has a simpler structure with unchanged functionality, the cost reduction also lowers the purchase cost for patients. Additionally, the detachable modular elbow design provides patients with the opportunity to purchase suitable elbows separately, avoiding the necessity of bundled purchases with the frame assembly in a field of forehead support and the elbow. The detachable quick-release magnetic buckle design also allows patients to buy different suitable fixing parts, further saving on the costs of purchase and replacement for users. The detachability of the parts and the assembly of the forehead support frame with fewer components also reduce maintenance costs for users.

### BRIEF DESCRIPTION OF THE DRAWINGS

FIG. 1 is a three-dimensional schematic diagram of a frame assembly in a field of forehead support in accordance with an embodiment;

FIG. 2 is an exploded view of a frame assembly in a field of forehead support in accordance with an embodiment;

FIG. 3 is a sectional schematic diagram of a frame assembly in a field of forehead support in accordance with an embodiment;

FIG. 4 is a usage schematic diagram of a frame assembly in a field of forehead support in accordance with an embodiment;

FIG. 5 is a schematic diagram showing the contact between the cushion pad and the forehead when a frame assembly in a field of forehead support is worn in accordance with an embodiment;

FIG. 6 is a schematic diagram of the connection between the forehead pad and the cushion pad in a frame assembly in a field of forehead support in accordance with an embodiment;

FIG. 7 is a schematic diagram of the angle α and the angle β at the connecting end parts of a frame assembly in a field of forehead support in accordance with an embodiment;

FIG. 8 is a schematic diagram of the angle γ of the support rod in a frame assembly in a field of forehead support in accordance with an embodiment;

FIG. 9 is a three-dimensional schematic diagram of the quick-release magnetic buckle in a frame assembly in a field of forehead support in accordance with an embodiment;

FIG. 10 is a disassembled schematic diagram of the quick-release magnetic buckle in a frame assembly in a field of forehead support in accordance with an embodiment;

FIG. 11 is a schematic diagram of the connection between the quick-release magnetic buckle and the forehead support frame in accordance with an embodiment;

FIG. 12 is a schematic diagram of the connection between the frame assembly in a field of forehead support and the mask in accordance with another embodiment;

FIG. 13 is a schematic diagram of the forehead support frame having three connection points in accordance with another embodiment;

FIG. 14 is a schematic diagram of the non-detachable cushion pad in a frame assembly in a field of forehead support in accordance with another embodiment;

FIG. 15 is a schematic diagram of another form of connection between the frame and the patient interface pad in accordance with another embodiment;

FIG. 16 is a schematic diagram of the connection between elbows with different exhaust port and the forehead support frame in accordance with an embodiment;

FIG. 17A and FIG. 17B are schematic diagrams of the connection between elbows with and without noise reduction and the forehead support frame in accordance with an embodiment;

FIG. 18A and FIG. 18B are schematic diagrams of the connection between the elbow and the forehead support frame after the elbow connects with different types of gas transmission tubes in accordance with an embodiment;

FIG. 19 is a schematic diagram of the exhaust port located on the frame in another embodiment;

FIG. 20 is a schematic diagram of other connection methods between the forehead support frame and the headgear, apart from the quick-release magnetic buckle, in accordance with an embodiment;

FIG. 21 is a schematic diagram of the direct connection between the elbow and the patient interface pad in accordance with an embodiment;

FIG. 22A, FIG. 22B, and FIG. 22C are schematic diagrams of the adjustable support rod in the forehead support frame in accordance with an embodiment;

FIG. 23A and FIG. 23B are schematic diagrams of the connection between the cushion pad and other comfort layers in accordance with an embodiment.

### DETAILED DESCRIPTION

To facilitate an understanding of this disclosure, a more comprehensive description will be provided below with reference to the relevant drawings. The drawings present typical embodiments of this disclosure. However, it should be understood that the disclosure can be implemented in many different forms and is not limited to the embodiments described herein. Rather, these embodiments are provided to make the disclosure more thorough and comprehensive.

Unless otherwise defined, all technical and scientific terms used herein have the same meaning as commonly understood by those of ordinary skill in the art to which this disclosure belongs. The terms used in the description of the disclosure herein are for the purpose of describing particular embodiments only and are not intended to limit the disclosure.

This disclosure aims to address the complexities, insufficient comfort, production difficulties, and high costs of existing forehead support frame assemblies on the market, which are configured to secure patient interface devices to the patient's face and assist in delivering gas to the patient's airway. It provides a frame assembly in a field of forehead support with a simpler structure, and it provides patients with a more comfortable wearing experience. The frame assembly in a field of forehead support designed in this disclosure not only optimizes various disadvantages of the existing designs but also offers several different structures of forehead support frame assemblies for patients to choose from according to their individual needs and preferences. This is a better technological advancement for users, producers, and the market. The reduction in material waste achieved by this disclosure also represents a sustainable, environmentally friendly design.

The following embodiments illustrate several structures of the frame assembly in a field of forehead support of this disclosure.

### Embodiment 1

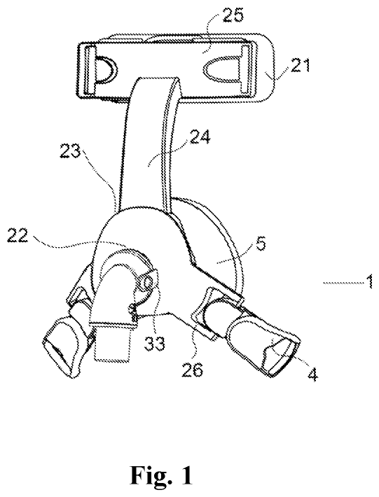

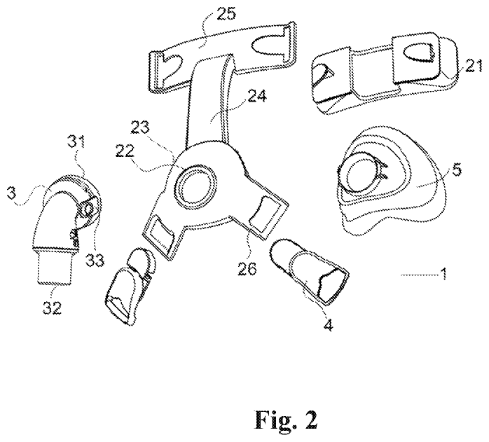

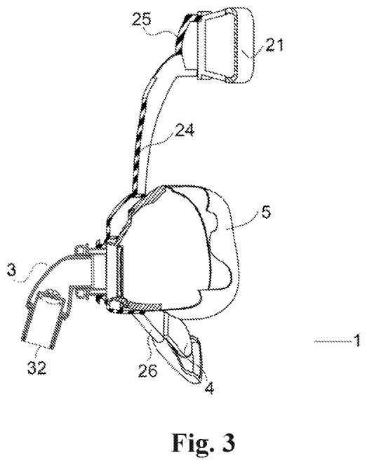

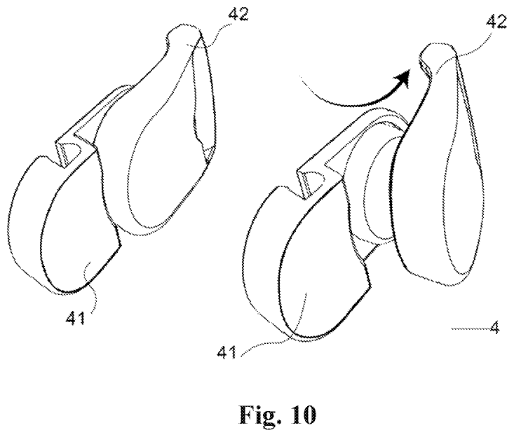

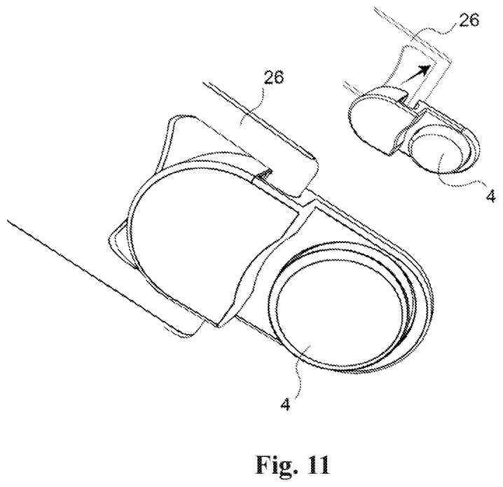

This embodiment provides a frame assembly 1 in the field of forehead support, configured to work in conjunction with other parts (such as a mask, a breathing tube, a respirator, etc.) that supply continuously pressurized respiratory gas to patients for the treatment of sleep-disordered breathing. This embodiment provides three-dimensional schematic diagrams, exploded views, front views, sectional schematic diagrams, usage diagrams, and detail display diagrams of the frame assembly in a field of forehead support 1, as shown in FIGS. 1-11. The frame assembly in a field of forehead support 1 shown in this embodiment mainly includes an elbow 3, a forehead support frame 2, a cushion pad 21, an exhaust port 34, and a quick-release magnetic buckle 4. The frame assembly in a field of forehead support 1 is configured to deliver pressurized gas from a ventilator into the patient's nasal airway for the treatment of obstructive sleep-disordered breathing.

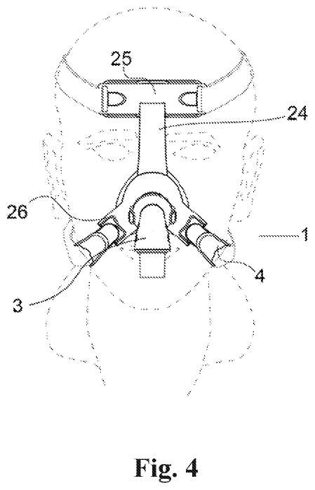

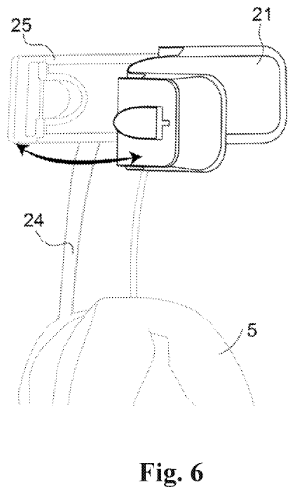

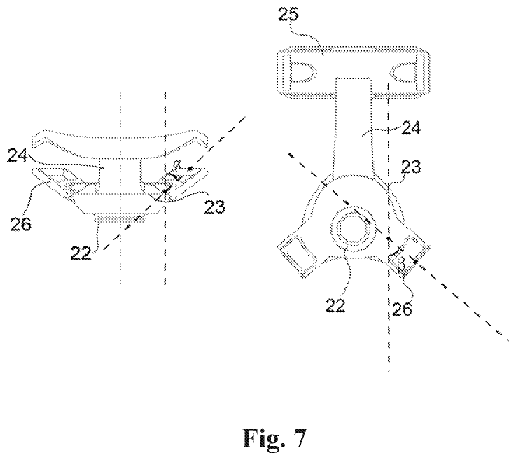

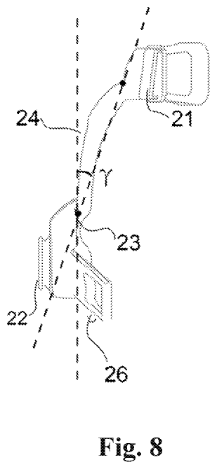

The frame assembly in a field of forehead support 1 includes a forehead support frame 2, including a first joint that forms a detachable connection with an elbow, a second joint away from the elbow, a wall formed between the first joint and the second joint, and a support rod extending upward from the wall between the first joint and the second joint to the forehead, along with a forehead pad connected to the support rod, configured to distribute the pressure of the patient interface pad on the face across the forehead during use. The first joint 22 of the forehead support frame 2 can have a wall protruding inward or outward to connect to the elbow 3. In use, at least part of the wall formed between the first joint 22 and the second joint 23 of the forehead support frame 2 contacts the outer wall of the patient interface pad 5. The wall formed between the first joint 22 and the second joint 23 of the forehead support frame 2 may have a snap-fitting to connect to the patient interface pad 5. The connection between the forehead support frame 2 and the patient interface pad 5 is formed by snap-fitting. Additionally, the forehead support frame 2 also has connecting end parts 26 on both sides extending at an angle (including angle α and angle β as shown in FIG. 7) towards the side nearer the patient's face to connect to fixing parts. The forehead pad 25 has at least one through opening to accommodate a headgear to form a connection (as shown in FIG. 4). Therefore, the forehead support frame 2 has at least three connection points to connect to a headgear. In this embodiment, there are four connection points. Two of the connection points are set on connecting end parts 26 that extend at an angle towards a side near the face on both sides of the forehead support frame 2, configured to connect to fixing parts or headgear. The fixing parts include quick-release magnetic buckles 4 and other components used for connection and fixation. Other connection points are configured and arranged on the forehead pad, specifically, the forehead pad 25 has through openings at both ends to accommodate a headgear to form a connection. Since the forehead pad 25 is configured to form a detachable connection with the cushion pad 21, there are various ways to connect to the forehead pad 25 with the cushion pad 21. In one of the connection methods, the forehead pad 25 has an opening to stabilize and accommodate a part of the cushion pad 21. The cushion pad 21 connects to the forehead support frame 2 by passing a part of itself through the opening in the forehead pad 25 (as shown in FIG. 6). The cushion pad 21 can also be connected to the forehead support frame 2 in other ways. Additionally, the entire forehead support frame 2 is made from the same material, which is plastic, such as high-density polyethylene, polypropylene, polycarbonate, polystyrene, etc. In the production and manufacturing process, the process is relatively simple and does not waste excess material. Since a part of the forehead support frame 2 needs to come into contact with the face, its structure and shape should conform to various characteristics of the face. For instance, because the forehead support frame 2 is fixed on the middle and lower part of the patient's face, around the mouth and nose, when in use, to ensure stability, the connection points between the forehead support frame 2 and the headgear should be distributed up and down. Therefore, the connecting end parts 26 on both sides of the forehead support frame 2 are designed below the wall between the first joint 22 and the second joint 23 and are set to protrude to facilitate the connection of the headgear or quick-release magnetic buckle 4. The length of the connecting end parts 26 on both sides of the forehead support frame 2 is at or between 1 to 5 cm. To further enhance its stability, the angle between the straight line where the connecting end parts 26 on both sides of the forehead support frame 2 are located, and a parallel line to the axis of the first joint 22 of the forehead support frame 2 is at or between 5 to 90° (as shown in FIG. 7, angle α). Moreover, the wall between the first joint 22 and the second joint 23 is configured to be arc-shaped to fit the patient interface pad 5. The angle between the straight line where the connecting end parts 26 on both sides of the forehead support frame 2 are located and a plumb line (a line perpendicular to the horizontal line) at its position is at or between 0 to 85° (as shown in FIG. 7, angle β). Since the distance from the tip of most people's noses to their foreheads is fixed, there is not a vertical plane but rather an angle from the nose tip to the forehead. To better adapt to the contours of the face, the height of the support rod 24 is set to be at or between 3 to 12 cm. The angle between the line connecting both ends of the support rod 24 on the forehead support frame 2 and the plumb line at its position is at or between 3 to 45° (as shown in FIG. 8, angle γ), ensuring that when the patient uses it, the forehead pad 25 fits snugly against the patient's forehead.

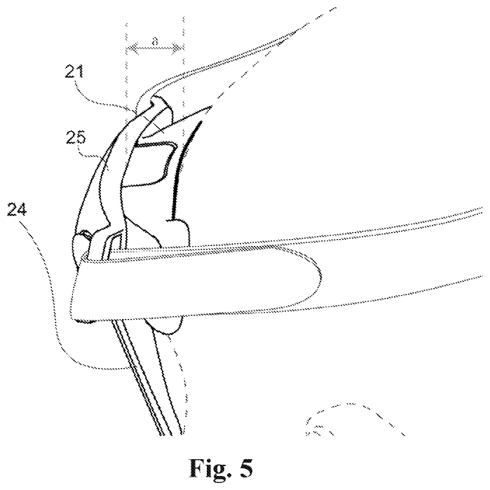

The frame assembly in a field of forehead support 1 also includes an elbow 3. The elbow 3 has a first interface 31 that forms a detachable connection with the first joint 22 of the forehead support frame 2. The elbow 3 also has a second interface 32 that connects to the gas transmission tube. The second interface is configured to receive ventilator airflow that flows through the gas transmission tube, and to deliver the ventilator airflow into the patient interface pad 5. In this embodiment, the elbow 3 includes a first interface 31 having a clamping structure 33, where the elbow 3 forms a detachable connection with the first joint 22 of the forehead support frame 2 through the clamping structure 33. The second interface 32 of the elbow 3 is configured to connect to the gas transmission tube. The elbow 3 can also have an exhaust port 34, configured to allow the continuous flow of exhaled gas to the external environment. Since the connection between the elbow 3 and the forehead support frame 2 is detachable, it allows for the selection of different elbows 3 to match the forehead support frame 2 without changing the forehead support frame 2, based on the patient's needs or preferences. Patients can choose the exhaust pressure of the elbow 3. They can also choose the elbow 3 with or without the noise reduction, as well as different diameters and lengths of the gas transmission tube to connect to the second interface 32 of the elbow 3. By replacing the elbow 3 with different apertures or numbers of exhaust ports, the airflow pressure entering the patient's airway can be adjusted, allowing patients to select an elbow 3 with different numbers of holes or apertures of holes based on their own respiratory resistance. Special patients with respiratory dysfunction and respiratory failure can also choose an elbow 3 without any holes for replacement. Additionally, patients can choose an elbow 3 with or without noise reduction. The second interface 32 of the elbow 3 can be connected to either a short tube or a gas transmission tube (by changing the size of the second interface 32 of the elbow 3): A short tube does not cause the patient to feel oppressed due to the large size or weight of the gas transmission tube while the gas transmission tube provides a larger gas storage capacity than a flexible hose, offering more flexibility in choice for users. The disclosure is aimed at allowing patients to autonomously choose an appropriate elbow 3, making continuous positive airway pressure therapy more comfortable. This method, with the same forehead support frame 2, matches different elbows, modularizing the frame assembly in a field of forehead support. The cushion pad 21 has a first end part 211 connected to the forehead pad 25, and a second end part 212 that contacts the patient's face. There is a certain distance between the first end part 211 and the second end part 212, configured to provide a certain level of cushioning to the patient's face during use. When the cushion pad 21 is placed on the forehead (as shown in FIG. 5), and the headgear is tightened, transferring a certain force to the forehead support frame 2, the presence of the cushion pad 21 becomes crucial. Without it, the relatively hard frame would concentrate the pressure, causing discomfort to the patient. However, the presence of the cushion pad 21 allows it to conform to the curvature of the patient's forehead and fit it, providing even distribution and absorption of some of the pressure, making it more comfortable to use. The distance between the first end part 211 and the second end part 212 of the cushion pad 21 is configured to provide extra space for cushion pad 21 to fit the forehead and disperse pressure. Specifically, the distance (as shown as ‘a’ in FIG. 5) between the first end part 211 and the second end part 212 of the cushion pad 21 is at or between 1 to 5 cm. The area of the cushion pad 21 that contacts the patient's face is no less than 6 cm² to ensure sufficient contact area and cushioning distance for the patient. The frame assembly in a field of forehead support also includes a detachable connection between the cushion pad 21 and the forehead support frame 2, facilitating cleaning and replacement by the patient. The thickness of the cushion pad 21 is at least 3 mm, and its material is selected from one or more material from foam material, silicone material.

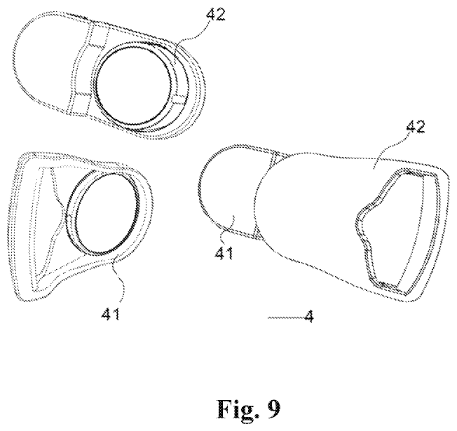

The quick-release magnetic buckle 4 includes connecting ends 41 that connect to the forehead support frame 2 and a magnetic attraction end 42. The quick-release magnetic buckle 4 (fixing parts) includes a protruding magnetic attraction structure that allows the forehead support frame 2 to be releasably connected and detached from the headgear. The connecting ends 41 of the quick-release magnetic buckle 4 have a snap-fit structure and form a detachable connection with the connecting end parts 26 on both sides of the forehead support frame 2. The specific method of use involves bringing one half of the magnetic buckle near the other half during connection, where they will attach due to the different magnetic poles, making connection relatively easy. Since the force from the connected headgear is broadly along the longer side of the quick-release magnetic buckle 4, the magnetic attraction end 42 has a protruding flange to ensure that the force that connects the headgear does not easily cause the magnetic buckle 4 to separate. For disassembly, one simply needs to gently lift one side of the quick-release magnetic buckle 4 in the normal direction of the protrusion on the magnetic attraction end 42, and the buckle easily comes apart (as shown in FIGS. 9-11). The design of the quick-release magnetic buckle 4 makes disassembly and connection exceptionally simple, suitable for use by the elderly or people with inconvenient hands. It saves at least half the time compared to disassembling or connecting steps of the connection components available on the current market, making it both time-saving and labor-saving.

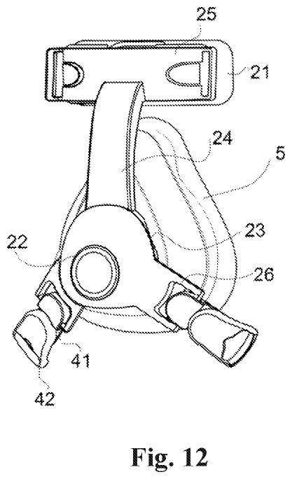

In another embodiment, the forehead support frame 2 of the frame assembly in a field of forehead support 1 can connect not only with a nasal mask but also with a full face mask, facilitating the modularization of the forehead support frame 2 (as shown in FIG. 12).

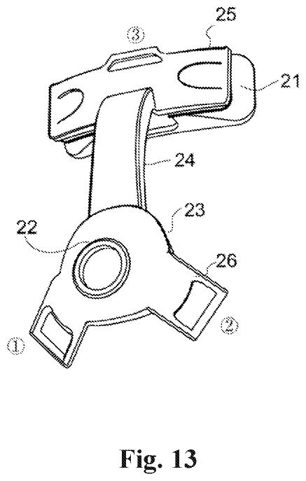

In another embodiment, the forehead support frame 2 in the frame assembly in a field of forehead support 1 has three connection points (as shown in FIG. 13).

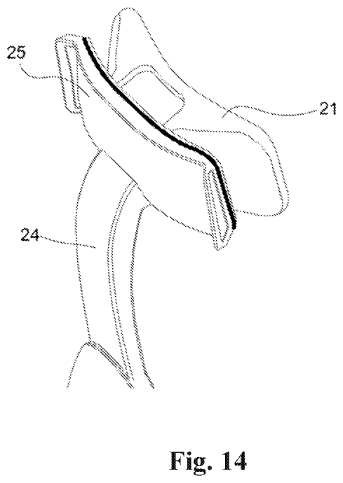

In another embodiment, the cushion pad 21 in the frame assembly in a field of forehead support 1 forms a non-detachable connection with the forehead pad 25 of the forehead support frame 2. The methods of connection can include ultrasonic bonding, welding, molding, gluing, etc. (as shown in FIG. 14, which illustrates a glued connection).

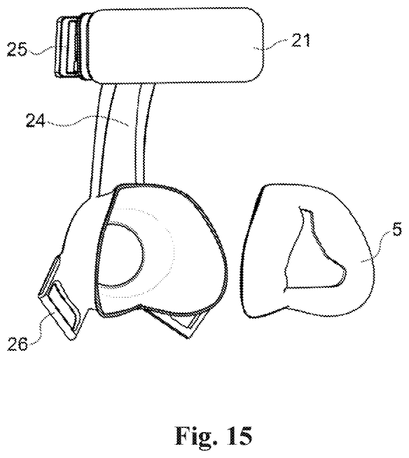

In another embodiment, the forehead support frame 2 is connected to a part of the patient interface pad 5. Specifically, it is connected to the patient interface pad through forms such as snaps, adhesive, or magnetic attraction, forming a detachable connection. This collectively creates a combined form of the patient interface pad 5 and the frame. FIG. 15 shows one of the snap-fit connections, specifically consisting of a flexible clip set on the patient interface pad 5, and a wall on the second joint 23 of the forehead support frame 2 with a corresponding groove for the flexible clip. Through their corresponding connection, the patient interface pad 5 is secured to the forehead support frame 2.

### Embodiment 2

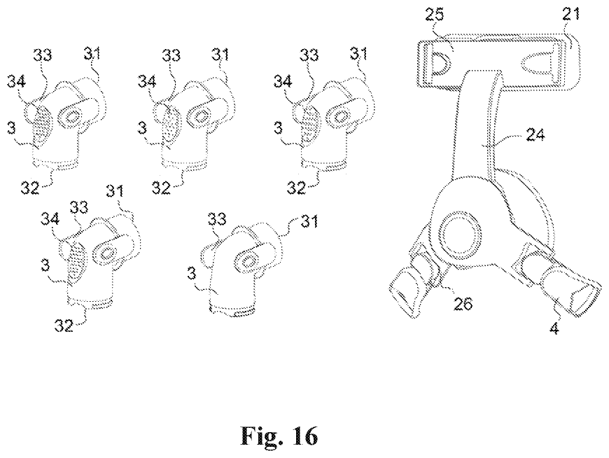

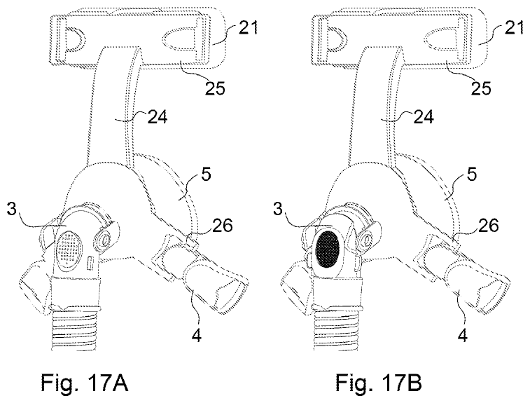

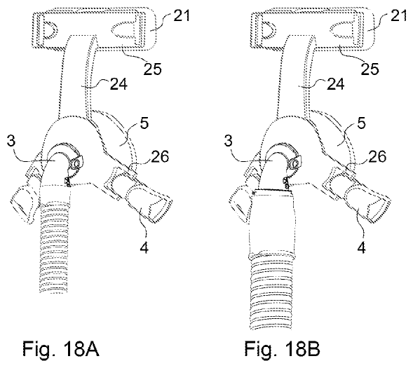

This embodiment provides a frame assembly in a field of forehead support 1 in the field of forehead support, as shown in FIGS. 16-18. This embodiment provides a three-dimensional schematic diagram of the frame assembly in a field of forehead support 1. In the embodiment of this disclosure, as shown in FIGS. 16-18, the difference from the frame assembly in a field of forehead support 1 in Embodiment 1 lies in the modularization of the elbow 3, and the frame assembly in a field of forehead support 1 has different forms of elbows 3 connected to the forehead support frame 2. Elbows 3 with varying apertures and numbers of holes, and hole shapes, corresponding to different air pressure sizes can be set. Based on feedback, different patients have varying preferences and adaptability to the airflow pressure during continuous positive airway pressure therapy. Therefore, different types of elbows 3 are configured to offer a range of choices for patients. Patients can choose different types of elbows 3 according to their needs and preferences for connection and use with the frame assembly in a field of forehead support 1. Special patients with respiratory dysfunction and respiratory failure can even choose an elbow 3 without holes for replacement (as shown in FIG. 16). Besides choosing elbows 3 with exhaust ports of different apertures or numbers, patients can also choose elbows 3 with or without noise reduction. Similar to patients' different preferences for air pressure, different patients have different preferences for noise reduction, and they can autonomously choose elbows 3 with or without noise reduction (as shown in FIG. 17, FIG. 17A shows an elbow without noise reduction function, and FIG. 17B shows an elbow with noise reduction function). Additionally, the choice of elbow 3 usually also involves the connection with the gas transmission tube. Gas transmission tubes come in different forms; larger tubes provide greater ventilation volume but can be bulky and give users a strong sense of oppression, while smaller tubes (such as short tubes) are more lightweight but offer less ventilation volume. Therefore, patients can also choose the appropriate elbow 3 based on its compatibility with the gas transmission tube, aimed at selecting an elbow 3 that is more suitable for the patient, making continuous positive airway pressure therapy more comfortable, and improving patient compliance with the treatment (as shown in FIG. 18, where FIG. 18A illustrates connection with a smaller gas transmission tube, and FIG. 18B shows connection with a larger gas transmission tube).

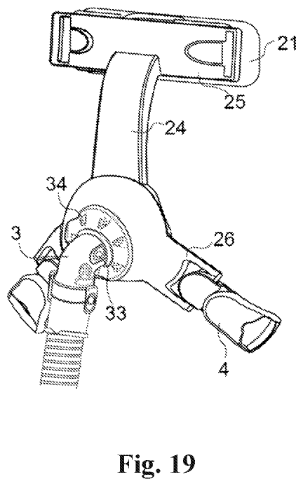

In another embodiment, the exhaust port 34 in the frame assembly in a field of forehead support 1 can also be set on the forehead support frame 2 itself (as shown in FIG. 19).

### Embodiment 3

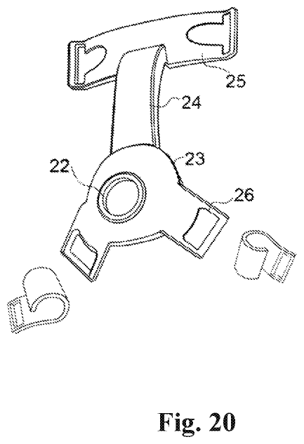

This embodiment provides a frame assembly in a field of forehead support 1 in the field of forehead support, as shown in FIG. 20. This embodiment provides a three-dimensional schematic diagram of the frame assembly in a field of forehead support 1. In the embodiment of this disclosure, as shown in FIG. 20, the difference from the frame assembly in a field of forehead support 1 in Embodiment 1 lies in that the fixing parts are not the quick-release magnetic buckle 4. In other words, the connection between the connecting end parts 26 on both sides of the forehead support frame 2 and the headgear is not made through the quick-release magnetic buckle 4. The connection between the connecting end parts 26 on both sides of the forehead support frame 2 and the headgear can also be achieved using components with hook structures or just directly using a hook and loop fastener. In this disclosure, the connection between the connecting end parts 26 on both sides of the forehead support frame 2 and the quick-release magnetic buckle 4 is made detachable, which means that patients can replace the magnetic buckle on the connecting end parts 26 of the forehead support frame 2 according to their own needs and preferences. While quick-release magnetic buckles 4 are convenient, they may not be the most suitable connection and separation method for some patients who are accustomed to direct connections between the frame and headgear or for patients who cannot be exposed to magnets (such as patients with metal implants). The replaceability of the product's components further increases the range of options for users, providing a suitable solution for those who are not accustomed to the quick-release magnetic buckle 4.

### Embodiment 4

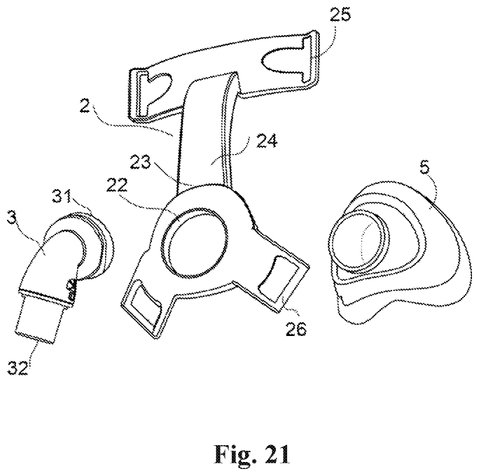

This embodiment provides a frame assembly in a field of forehead support 1 in the field of forehead support, as shown in FIG. 21. This embodiment provides a three-dimensional schematic diagram of the frame assembly in a field of forehead support 1. In the embodiment of this disclosure, as shown in FIG. 21, the difference from the frame assembly in a field of forehead support 1 in Embodiment 1 lies in setting the bend 3 directly connected to the patient interface pad 5, using the forehead support frame 2 as a modular component, and the first joint and the second joint 23 of the forehead support frame 2 both have smooth-walled openings. When in use, the forehead support frame 2 is clamped and constrained between the elbow 3 and the patient interface pad 5. The advantage of using the forehead support frame 2 as a modular component is the ability to adapt and create more diverse combinations. The same forehead support frame 2 can be adapted to different elbows 3 and patient interface pads 5, expanding the range of choices for patients. This allows the forehead support frame 2 to circulate in the market for a longer time, further simplifies the structure of the forehead support frame 2, eliminates the need to consider the compatibility between multiple components in production, streamlines the production process, and saves production and time costs. Additionally, the modular forehead support frame 2 components can quickly adapt to the market, facilitating rapid updates and iterations of the product in response to market demands.

### Embodiment 5

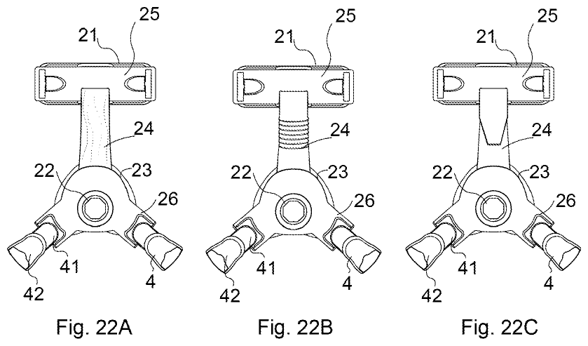

This embodiment provides a frame assembly in a field of forehead support 1 in the field of forehead support, as shown in FIG. 22. This embodiment provides a three-dimensional schematic diagram of the frame assembly in a field of forehead support 1. In the embodiment of this disclosure, as shown in FIG. 22, the difference from the frame assembly in a field of forehead support 1 in Embodiment 1 lies in setting the support rods 24 in the forehead support frame 2 to be adjustable. Due to the different heights and protrusions of patients' foreheads, and since the forehead support frame 2 is involved in coordinating with the patient's face, the distances between various parts of the forehead support frame 2 can only be estimated based on standard facial measurements. However, this approach may not fit all patients. Therefore, it is necessary to design an adjustable forehead support frame 2. Its greatest uncertainty lies in the height and protrusion of the patient's forehead. Therefore, it is appropriate to set the adjustability on the support rod 24 of the forehead support frame 2. There can be multiple forms of adjustment methods. Support rod 24 and forehead pad 25 are structurally configured to be adjustable and are mechanically adjusted through the structure (as shown in FIG. 22C). This method doesn't require multiple materials but can be relatively complex and challenging in manufacturing. This method does not require the use of multiple materials, but it is relatively complex and difficult in terms of manufacturing. Another method involves adjustment through the properties of the material or its special structure. The support rod 24, either entirely or a section of it, can be made of a flexible material that can be stretched. During use, it is manually stretched to set the distance between the forehead pad 25 and the second joint 23 of the forehead support frame 2, to achieve a range suitable for the patient (as shown in FIGS. 22A and 22B).

### Embodiment 6

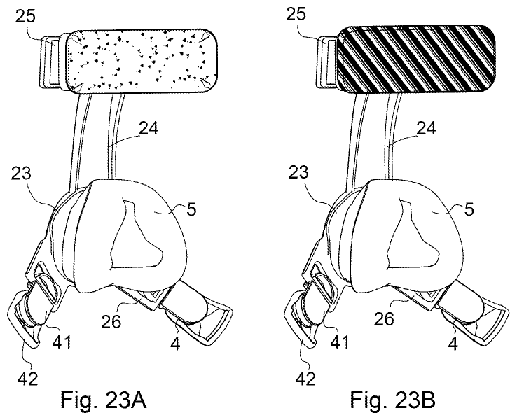

This embodiment provides a frame assembly in a field of forehead support 1 in the field of forehead support, as shown in FIG. 22. This embodiment provides a three-dimensional schematic diagram of the frame assembly in a field of forehead support 1. In the embodiment of this disclosure, as shown in FIG. 22, the difference from the frame assembly in a field of forehead support 1 in Embodiment 1 lies in that a comfort layer is set on the cushion pad 21 in the frame assembly in a field of forehead support 1. In some embodiments, the material of the cushion pad 21 is made of silicone, which is relatively less breathable. Additionally, when silicone comes into contact with the patient's face, it tends to slip when the patient sweats, or some patients may need a more comfortable cushion pad 21. Therefore, a layer of textile material or foam material can be added on top of the silicone for breathability and moisture absorption. This ensures that patients do not feel stuffy, hot, or uncomfortable during use. The addition of the comfort layer also makes the cushion pad 21 softer and more comfortable, enhancing the comfort of the treatment. The comfort layer also acts as a barrier, reducing direct contact between the face and the cushion pad 21. This avoids potential irritation and skin abrasion for users who are sensitive to skin, allergic to silicone rubber, or dislike the existing cushion pad 21. Adding the comfort layer on the cushion pad 21 provides patients with a more comfortable wearing experience. The comfort layer can be made of foam material (as shown in FIG. 23A), textile material (as shown in FIG. 23B), or other comfortable materials. Moreover, the addition of the comfort layer makes it easier for patients to clean the frame assembly in a field of forehead support 1, helping to maintain its cleanliness and hygiene, and ensuring the effectiveness of the treatment.

The various technical features of the embodiments described above can be combined arbitrarily. For brevity, not all possible combinations of these technical features in the aforementioned embodiments are described. However, as long as there is no contradiction in the combination of these technical features, they should be considered within the scope documented in this specification.

Implementing the frame assembly in the field of forehead support in the disclosure provides at least the following beneficial effects:

1. In terms of comprehensive aspects, compared to existing forehead support frames on the market, the forehead support frame disclosed has superior highlights in all aspects. It is more advantageous and belongs to a more competitive frame assembly in a field of forehead support. Referencing the design philosophy from the Tesla automotive design field, the disclosure simplifies the frame and heavily incorporates modular design. The forehead support frame is treated as a separate component. Under the premise of ensuring and optimizing its functionality, the forehead support frame is made more convenient and comfortable. Specifically, a. compared to the existing forehead support frames like the Mirage Activa™ LT Nasal CPAP Mask, ComfortGel Blue, and Ultra Mirage™ II Nasal CPAP Mask, they require connection to larger patient interface pads, appear bulky visually and in use, exert a more serious sense of pressure on the patient, and are likely to cause discomfort. Further, the larger patient interface pad encompasses a greater area of the patient's face, meaning that the discrepancy in level in how the relatively bigger patient interface pad fits around different parts of the face is greater, making it more prone to air leakage and thus reducing the effectiveness of the treatment. In the disclosure, the forehead support frame connects to a smaller patient interface pad, making it more patient-friendly and less prone to air leakage issues. It uses less material and is smaller in size, which makes it easier to carry and transport. It also includes quick-release magnetic buckles at the connection ends where the frame joins the headgear on both sides, making the installation and removal of the frame much simpler and providing a more convenient user experience for patients. The quick-release magnetic buckles are connected to the connecting end parts of the sides of the forehead support frame through a snap-fitting. The buckles have protrusions to store magnets and during connection, the buckles will be connected by the attraction from different magnetic poles on the opposite side. Due to the protruding and connection to corresponding grooves on the other side, they have a baffle in the direction of the force, making them unlikely to fall off. To detach, one simply needs to pull one side slightly in a perpendicular direction for easy removal. This method is time-saving and labor-saving, as the quick-release magnetic buckles are designed for a detachable connection with the connecting end parts on both sides of the forehead support frame. Patients can choose to use different quick-release connectors or not use them at all, according to their preferences or needs, offering a variety of different options. If there is a gas leak in the frame assembly in a field of forehead support, the quick-release design of the magnetic buckle facilitates rapid adjustment of the frame assembly in a field of forehead support by the patient. The design of the quick-release magnetic buckle also makes the disassembly and installation steps more straightforward and clear, which is beneficial for elderly patients or those with less dexterity in their hands to install and remove. The disclosure, compared to the existing forehead support frames mentioned above, reduces the time required for disassembly or installation by at least half. Regarding the overall design of the forehead support frame, the forehead support frame in the disclosure creates a form that is more straightforward, mostly composed of smooth and neat walls. By presenting the forehead support frame as a simpler module, it not only provides advantages in terms of cleaning and patient use but also in production. b. Referencing the existing ResMed Mirage™ FX, Eson™ Nasal CPAP Mask, and Eson™ 2 Nasal CPAP Mask, a part of the forehead support frame where it contacts the patient's forehead in these designs is a rigid part. Although the rigid part is shaped to curve towards the face side to fit the patient's forehead as closely as possible, it is not entirely effective. Since the human forehead is not a perfectly smooth curve, the forehead pad cannot fully conform to the patient's forehead to distribute force for more comfortable use for patients. Furthermore, people have varying degrees of forehead protrusion, and the existing forehead pads of these forehead support frames cannot accommodate the degree of forehead protrusion of different users. When pressure is concentrated on the forehead, some patients may feel very uncomfortable. Additionally, the rigid parts usually do not have the ability to absorb shocks or compression. When the rigid part comes into direct contact with the skin, any external force is directly transmitted to the skin, and the lack of breathability in the rigid parts can easily lead to moisture accumulation, causing discomfort to the patient's skin. The disclosure utilizes a cushion pad placed at the forehead pad area, which on the one hand, adapts to the curve or irregular shape of the forehead. The flexible material evenly distributes the pressure brought about by the headgear fixation, preventing pressure concentration in specific areas, like higher protrusions on the forehead, thereby avoiding pain or discomfort. The flexible material absorbs a certain amount of force, thus reducing the sensation of pressure on the patient's forehead. The gentle touch of the flexible material provides a more comfortable wearing experience for the patient. Furthermore, the present disclosure has quick-release magnetic buckles, which make it more convenient and easy to disassemble and install, saving time and effort. c. The existing Mirage™ FX forehead support frame has a complex structure. Though it offers convenience in terms of functionality, the complex structure and manufacturing process of the Mirage™ FX result in high production and processing costs. It uses silicone and rigid materials and is constructed through a secondary injection molding process. Additionally, its exhaust port is located on the matching patient interface pad. This production method leads to a higher defect rate and increased costs. In special circumstances, the supply of Mirage™ FX may become relatively difficult, leading to prolonged periods of product unavailability. The forehead support frame of this disclosure is entirely made of one material, which simplifies the structure on the premise of ensuring functionality. It mostly consists of smooth walls. Furthermore, the exhaust port of the frame assembly in a field of forehead support of the disclosure is located on the elbow. The elbow forms a detachable connection with the forehead support frame, reducing the defect rate and production cost of the frame assembly in a field of forehead support. This enhances the quality of the product and has a simple processing, preventing stock shortages.

2. The frame assembly in a field of forehead support of this disclosure simplifies the product structure on the premise of ensuring its functional integrity, thereby improving supply chain efficiency, and reducing production costs and risks. This makes the frame assembly in a field of forehead support of the disclosure more competitive in the market. The forehead support frame shown in the disclosure can be broadly divided into four parts. It is integrally injection-molded from one material, and the four parts include, the central part that includes the first joint, the second joint, and the wall connecting the first joint and the second joint, and the central part is configured to connect to the patient interface pad; the forehead pad part; the support rod part connecting the forehead pad with the central part; and the connecting end parts on both sides. The outer walls of all four parts are smooth and uniform without any complex structure such as protrusions or indentations, making the form simple and the volume relatively smaller, allowing for more simple and more efficient production for molds, reducing manpower, equipment, and energy costs during manufacturing. The reduction in costs and increased efficiency allow for quicker adjustments to rapidly respond to and meet changes in market demand, enabling more rapid innovation. The simplicity of the structure makes the production process easier to manage and control, ensuring product quality. Moreover, the reduction in parts and the use of modular components can effectively lower inventory variety, reduce the risk of excess stock, and simplify the complexity of material management. In summary, this disclosure better meets customer needs, enhances product maintainability and life-cycle value, and improves supply chain efficiency. Additionally, the simplified structure leads to reduced material usage and the abandonment rate of waste, positively impacting environmental protection and climate change.

3. The disclosure includes a quick-release elbow that forms a detachable connection with the forehead support pad, modularizing the elbow. Patients can choose different elbows to connect to the forehead support frame according to their preferences and needs. Therefore, different types of patients are allowed to replace different elbows to adapt to varying degrees of airflow pressure and to accommodate tubes of different diameters and lengths. According to some opinions from CPAP forums and Amazon customer reviews, the disclosure understands that different patients have different requirements for the pressure of the airflow entering their airways. Moreover, some patients find a 22 mm gas transmission tube to be somewhat cumbersome and oppressive for the nasal mask. Patients also have varying needs regarding the length of the tube. Therefore, in this disclosure's frame assembly in a field of forehead support, the exhaust port is set on the detachable elbow. If a patient cannot obtain the optimal airflow pressure size from the ventilator to meet their needs, they can adjust the airflow pressure entering their airway by replacing the elbow with different apertures or numbers of exhaust ports. Patients can choose different elbows with different apertures or numbers of exhaust ports according to their own respiratory resistance. For special patients with respiratory dysfunction and respiratory failure, elbows without holes can also be chosen and replaced. In addition to choosing elbows with different apertures or numbers of exhaust ports, patients can also choose elbows with or without noise reduction. Generally, elbows with noise reduction and those without are two different types of elbows. The modular design offers a variety of choices for elbows, allowing users who prefer noise reduction or no noise reduction to purchase and replace them according to their preferences or needs. Additionally, the second interface of the elbow, which connects to the tube, also comes in different sizes to connect to tubes of varying diameters or lengths, such as short tubes or gas transmission tubes. This is intended to allow patients to independently choose the elbow that best suits them, making continuous positive airway pressure (CPAP) therapy more comfortable.

4. The frame assembly in a field of forehead support provided by this disclosure reduces user costs in multiple aspects. Because the forehead support frame of this disclosure has a simpler structure with unchanged functionality, the cost reduction also lowers the purchase cost for patients. Additionally, the detachable modular elbow design provides patients with the opportunity to purchase suitable elbows separately, avoiding the necessity of bundled purchases with the frame assembly in a field of forehead support and the elbow. The detachable quick-release magnetic buckle design also allows patients to buy different suitable fixing parts, further saving on the costs of purchase and replacement for users. The detachability of the parts and the assembly of the forehead support frame with fewer components also reduce maintenance costs for users.

The technical features of the embodiments described above can be combined in any manner. To keep the description concise, not all possible combinations of the technical features in the above embodiments are described. However, as long as there is no contradiction in these combinations of technical features, they should be considered within the scope of this specification.

The embodiments described above only represent a few of the possible implementations of this disclosure and are described in specific and detailed terms, but should not be construed as limiting the scope of the patent for the disclosure. It should be noted that, for those skilled in the art, various modifications and improvements can be made without departing from the concept of this disclosure, and these are also within the scope of protection of this disclosure. Therefore, the scope of protection of the patent for this disclosure should be determined by the attached claims.

It must be noted that as used herein and in the appended claims, the singular forms “a”, “an”, and “the” include their plural equivalents, unless the context clearly dictates otherwise.

1. A frame assembly in a field of forehead support, for use in conjunction with other parts to supply continuous pressurized breathing gas to airways for treatment of sleep breathing disorders, wherein the frame assembly comprises:
a forehead support frame, comprising a first joint that forms a detachable connection with an elbow, a second joint away from the elbow, a wall formed between the first joint and the second joint, a support rod extending upward from the wall between the first joint and the second joint to a forehead, and a forehead pad connectable to the support rod, wherein the forehead pad is configured to distribute pressure of a patient interface pad on a face across the forehead during use;
wherein the forehead support frame further includes connecting heads on both sides that extend at an angle towards a side near the face and configured to connect to fixing parts,
wherein the entire forehead support frame is made of a same material;
the elbow comprising a first interface formed between an outwardly extending flange and a first end of the elbow with a clamping structure on opposite sides of the elbow,
wherein the first joint has a central aperture and a groove extending around an exterior surface of the first joint,
wherein the first interface of the elbow extends into the central aperture of the first joint,
wherein the elbow forms a detachable connection with the first joint of the forehead support frame through the clamping structure,
wherein the elbow includes an exhaust port that is configured to allow a continuous flow of exhaled gas to an external environment,
wherein an angle between a line connecting both ends of the support rod of the forehead support frame and a plumb line at its position is between 3° to 45°, and
wherein an angle between a straight line where the connecting heads on both sides of the forehead support frame are located and a parallel line to an axis of the first joint of the forehead support frame is between 5° to 90°.

2. The frame assembly in a field of forehead support according to claim 1, wherein the frame assembly further comprises a cushion pad that forms a detachable connection with the forehead support frame.

3. (canceled)

4. The frame assembly in a field of forehead support according to claim 1, wherein an angle between the straight line where the connecting heads on both sides of the forehead support frame are located and a plumb line at its position is between 0 to 85°.

5. The frame assembly in a field of forehead support according to claim 1, wherein the wall formed between the first joint and the second joint of the forehead support frame comprises a snap-fitting to connect to the patient interface pad.

6. A frame assembly in a field of forehead support, for use in conjunction with other parts to supply continuous pressurized breathing gas to airways for treatment of sleep breathing disorders, wherein the frame assembly comprises:
a forehead support frame, comprising a first joint that forms a detachable connection with an elbow, a second joint away from the elbow, a wall formed between the first joint and the second joint, a support rod extending upward from the wall between the first joint and the second joint to a forehead, and a forehead pad connectable to the support rod, wherein the forehead pad is configured to distribute pressure of a patient interface pad on a face across the forehead during use;
wherein the forehead support frame further includes connecting heads on both sides that extend at an angle towards a side near the face and configured to connect to fixing parts;
wherein the elbow comprises a first interface formed between an outwardly extending flange and a first end of the elbow with a clamping structure on opposite sides of the elbow,
wherein the first joint has a central aperture and a groove extending around an exterior surface of the first joint,
wherein the first interface of the elbow extends into the central aperture of the first joint,
a cushion pad, comprising a first end part connectable to the forehead pad, and a second end part that contacts the face, which is configured to provide cushioning to the face during use;
wherein the fixing parts are quick-release magnetic buckles, comprising connecting ends that connect to the forehead support frame and a magnetic attraction end,
wherein the quick-release magnetic buckles are configured to have a protruding magnetic attraction structure to allow the forehead support frame to be releasably connectable and detachable from a headgear,
wherein an angle between a line connecting both ends of the support rod of the forehead support frame and a plumb line at its position is between 3° to 45°, and
wherein an angle between a straight line where the connecting heads on both sides of the forehead support frame are located and a parallel line to an axis of the first joint of the forehead support frame is between 5° to 90°.

7. The frame assembly in a field of forehead support according to claim 6, wherein the cushion pad forms a detachable connection with the forehead pad of the forehead support frame.

8. The frame assembly in a field of forehead support according to claim 6, wherein an area of the cushion pad in contact with the face is no less than 6 cm².

9. The frame assembly in a field of forehead support according to claim 6, wherein a thickness of the cushion pad is at least 3 mm.

10. The frame assembly in a field of forehead support according to claim 6, wherein a material of the cushion pad includes one or more of foam material or silicone material.

11. A frame assembly in a field of forehead support, for use in conjunction with other parts to supply continuous pressurized breathing gas to airways for treatment of sleep breathing disorders, wherein the frame assembly comprises:
a forehead support frame, comprising a first joint that forms a detachable connection with an elbow, a second joint away from the elbow, a wall formed between the first joint and the second joint, a support rod extending upward from the wall between the first joint and the second joint to a forehead, and a forehead pad connectable to the support rod, wherein the forehead pad is configured to distribute pressure of a patient interface pad on a face across the forehead during use,
wherein the forehead pad includes at least one through opening to accommodate a headgear to form a connection;
wherein the forehead support frame further includes connecting heads on both sides that extend at an angle towards a side near the face to connect to fixing parts;
wherein the elbow comprises a first interface formed between an outwardly extending flange and a first end of the elbow with a clamping structure on opposite sides of the elbow,
wherein the first joint has a central aperture and a groove extending around an exterior surface of the first joint,
wherein the first interface of the elbow extends into the central aperture of the first joint,
a cushion pad, including a first end part connectable to the forehead pad and a second end part that contacts the face,
wherein, there is a certain distance between the first end part and the second end part, configured to provide cushioning to the face during use;
wherein an area of the cushion pad that contacts the face is no less than 6 cm²,
wherein an angle between a line connecting both ends of the support rod of the forehead support frame and a plumb line at its position is between 3° to 45°, and
wherein an angle between a straight line where the connecting heads on both sides of the forehead support frame are located and a parallel line to an axis of the first joint of the forehead support frame is between 5° to 90°.

12. The frame assembly in a field of forehead support according to claim 11, wherein the cushion pad forms a non-detachable connection with the forehead pad of the forehead support frame.

13. The frame assembly in a field of forehead support according to claim 11, wherein the distance between the first end part and the second end part of the cushion pad is between 1 cm to 5 cm.

14. (canceled)

15. The frame assembly in a field of forehead support according to claim 11, wherein the forehead pad includes an opening that stabilizes and accommodates a part of the cushion pad.

16. A frame assembly in a field of forehead support, for use in conjunction with other parts to supply continuous pressurized breathing gas to airways for treatment of sleep breathing disorders, wherein the frame assembly comprises:
a forehead support frame, comprising a first joint that forms a detachable connection with an elbow, a second joint away from the elbow, a wall formed between the first joint and the second joint, a support rod extending upward from the wall between the first joint and the second joint to a forehead, and a forehead pad connectable to the support rod, wherein the forehead pad is configured to distribute pressure of a patient interface pad on a face across the forehead during use;
wherein the forehead support frame includes at least three connection points to connect to a headgear,
wherein two of the connection points are provided on connecting heads that extend at an angle towards a side near the face on both sides of the forehead support frame, wherein the connection points are configured to connect to fixing parts or the headgear;
the elbow, comprising a first interface formed between an outwardly extending flange and a first end of the elbow with a clamping structure on opposite sides of the elbow,
wherein the first joint has a central aperture and a groove extending around an exterior surface of the first joint,
wherein the first interface of the elbow extends into the central aperture of the first joint,
wherein the elbow forms the detachable connection with the first joint of the forehead support frame through the clamping structure,
wherein the elbow includes an exhaust port, configured to allow a continuous flow of exhaled gas to an external environment,
wherein an angle between a line connecting both ends of the support rod of the forehead support frame and a plumb line at its position is between 3° to 45°, and
wherein an angle between a straight line where the connecting heads on both sides of the forehead support frame are located and a parallel line to an axis of the first joint of the forehead support frame is between 5° to 90°.

17. The frame assembly in a field of forehead support according to claim 16, wherein the frame assembly further comprises a cushion pad that forms a detachable connection with the forehead support frame.

18. The frame assembly in a field of forehead support according to claim 16, wherein a length of the connecting heads on both sides of the forehead support frame is between 1 cm to 5 cm.

19. The frame assembly in a field of forehead support according to claim 16, wherein an area of the cushion pad that contacts the face is no less than 6 cm².

20. (canceled)

21. The frame assembly in a field of forehead support according to claim 16, wherein a height of the support rod is between 3 cm to 12 cm.
